(19) United States
(12) Patent Application Publication
(10) Pub. No.: US 2008/0038226 A1
(43) Pub. Date: Feb. 14, 2008

(54) Culture Methods for Cryptosporidium

(75) Inventors: Nawal Hijjawi, Rivervale (AU); Andrew R.C. Thompson, Roleystone (AU); Una M. Ryan, South Fremantle (AU)

Correspondence Address: MARSHALL, GERSTEIN & BORUN LLP, 233 S. WACKER DRIVE, SUITE 6300, SEARS TOWER, CHICAGO, IL, 60606, US

(73) Assignees: Murdoch University, Modoch (AU); Sydney Water Corporation, Sydney (AU)

(21) Appl. No.: 10/590,817

(22) PCT Filed: Feb. 25, 2005
(86) PCT No.: PCT/AU05/00262

§ 371 Date: Jun. 29, 2007

(30) Foreign Application Priority Data
Feb. 26, 2004 (AU) 20049000960

(51) Int. Cl. A61K 35/68; A61P 31/00; C12N 1/10; C12Q 1/04; C12Q 1/68

(52) U.S. Cl. 424/93.1; 435/258.1; 435/34; 435/6

(57) ABSTRACT

A host-cell free method for culturing Cryptosporidium comprising the step of introducing Cryptosporidium, at a first lifecycle stage, into a host-cell free medium under conditions which enable the Cryptosporidium to progress to a second lifecycle stage.

### FIELD OF THE INVENTION

The present invention relates to propagation systems for Cryptosporidium and more particularly to host-cell free systems and methods. The present invention also relates to methods for detecting Cryptosporidium, to host-cell free media and to the use of Cryptosporidium prepared using the methods and systems including their use in the preparation of Cryptosporidium vaccines and therapeutics.

### BACKGROUND ART

Intestinal protozoa cause a variety of clinical and economically important diseases in human and animals. Examples of known pathogenic intestinal protozoa include Giardia, Trichomonads, Histomonas, Spironucleus, Entamoeba, Coccidia, Sarocystis and Cryptosporidium.

Cryptosporidium is an Apicomplexan protozoan parasite that invades the intestinal epithelial cells of humans and various mammalian hosts, domesticated farm animals and poultry. In humans, the parasite infects the microvillus border of the intestinal epithelium, causing acute, self-limiting diarrhoea in immunocompetent individuals, and a chronic, life-threatening disease in immunocompromised patients. C. parvum demonstrates broad mammalian host specificity, infecting humans through direct human contact and via zoonotic transmission and has become a leading cause of diarrhoea in calves.

At least two species of Cryptosporidium infect cattle. C. parvum is characterized by small-type oocysts (5.0×4.5 μm) and primarily infects the intestine of young calves, resulting in considerable economic losses in the cattle industry and water-borne outbreaks of diarrheal disease in human populations. C. andersoni is a recently renamed species characterized by larger oocysts (9.7×5.6 μm) that infects the abomasum (fourth division of the stomach in ruminant animals) of cattle. To date, no effective treatment for cryptosporidiosis is available.

Cryptosporidium oocysts are transmitted by the fecal-oral route, and can be transmitted through contaminated water supplies and public swimming pools in endemic regions.

Following ingestion by a suitable host, Cryptosporidium oocysts excyst in the presence of host bile salts and pancreatic enzymes. The resulting sporozoites infect intestinal epithelial cells and differentiate into trophozoites. The trophozoites multiply asexually to produce type I schizonts containing about 6-8 merozoites. These merozoites can invade additional cells upon rupture of the schizonts. Merozoites may continue to develop into type I schizonts or form type II schizonts, which further differentiate into either male microgamonts or female macrogamonts. Male microgamonts release microgametes that fertilize the female macrogamont, resulting in an oocyst. Thick-walled oocysts pass through the digestive tract of the host while thin-walled oocysts likely reinfect the host.

Oocysts and sporozoites can be obtained from infected animals (e.g., calves) in large quantities. However, the handling and maintenance of infected animals constitute a risk of infection to humans. In addition, obtaining parasites from infected animals presents difficulties in terms of standardization of assays and experimental reproducibility.

The ability to propagate Cryptosporidium in vitro was first achieved in the endodermal cells of the chorioallantoic membrane (CAM) of chicken embryos. Numerous subsequent reports of the cultivation of the parasite in different cell lines followed, with varying degrees of success in terms of the development of Cryptosporidium life cycle stages. Although improvements in the in vitro culture of Cryptosporidium have occurred in recent years, continuous culture and efficient life cycle completion (oocyst production) have only recently been achieved in vitro, with long term maintenance of the life cycle of three species of Cryptosporidium (C. parvum, C. hominis and C. andersoni) now possible.

Cryptosporidium cell culture systems have aided many aspects of Cryptosporidium research. However, current culture methods need host cells and thus are relatively complex. Furthermore, overgrowth and aging of host cells can prevent perpetuation of the Cryptosporidium life cycle in vitro. The present invention seeks to overcome or at least ameliorate one or more of the problems attendant with the prior art.

### SUMMARY OF THE INVENTION

The present invention provides a host-cell free method for culturing Cryptosporidium comprising the step of introducing Cryptosporidium, at a first lifecycle stage, into a host-cell free medium under conditions that enable the Cryptosporidium to progress to a second lifecycle stage.

The method of the present invention can be used to culture Cryptosporidium through its complete lifecycle. Thus, the present invention also provides a host-cell free method for culturing Cryptosporidium comprising the step of introducing Cryptosporidium, at a first lifecycle stage, into a host-cell free medium under conditions that enable the Cryptosporidium to complete its lifecycle.

The present invention enables large amounts of Cryptosporidium to be conveniently produced without using host cells. Thus, the present invention also provides a host-cell free method for producing Cryptosporidium biomass from an initial inoculum of Cryptosporidium comprising the steps of: (i) putting the inoculum into a host cell free medium; and (ii) culturing the Cryptosporidium to increase the Cryptosporidium biomass.

Oocysts are a particularly useful starting material for the culture of Cryptosporidium. Thus, the present invention also provides a host-cell free method for culturing Cryptosporidium comprising the steps of:
a. isolating Cryptosporidium oocysts;
b. excysting the isolated oocysts;
c. resuspending the excysted oocysts in a host-cell free culture medium;
d. incubating the culture prepared in step (c) under suitable conditions; and
e. harvesting oocysts from the medium.

The media used in the present invention also represent an aspect of the invention. Thus, the present invention also provides a host cell free medium capable of maintaining Cryptosporidium or enabling the progress of Cryptosporidium through its lifecycle, the medium comprising a buffered and balanced combination of inorganic salts, amino acids, vitamins and additional constituents.

The culture methods of the present invention enable the production of Cryptosporidium more amenable to other applications. Thus, the present invention also provides a method for preparing an immunogenic preparation comprising at least one Cryptosporidium antigen, the method comprising the steps of: (i) introducing Cryptosporidium, at a first lifecycle stage, into a host-cell free medium under conditions which enable the Cryptosporidium to progress to a second lifecycle stage; (ii) isolating the Cryptosporidium at the second lifecycle stage; and (iii) preparing a therapeutic preparation using the Cryptosporidium isolated from step (ii).

Therapeutic compositions comprising a therapeutically effective amount of Cryptosporidium cultured according to the invention are also described herein as are methods of preventing or treating a disease associated with Cryptosporidium infection in a subject.

The present invention further provides methods for detecting Cryptosporidium in a sample comprising the steps of: (i) subjecting the sample to culture using a host cell free medium; and (ii) detecting the Cryptosporidium.

The invention still further provides a method for culturing Cryptosporidium comprising the steps of:
(a) introducing a stage in the life cycle of Cryptosporidium into culture media selected from a maintenance medium or a biphasic medium in the absence of host cells; and
(b) culturing the Cryptosporidium.

The invention also provides a culture method comprising the steps of:
(a) isolating Cryptosporidium oocysts;
(b) excysting the isolated oocysts;
(c) recovering the excysted oocysts;
(d) resuspending the recovered oocysts in maintenance media;
(e) incubating the culture prepared in step (d); and
(f) recovering the oocysts.

The invention still further provides a culture method comprising the steps of:
(a) isolating Cryptosporidium oocysts;
(b) excysting the isolated oocysts;
(c) recovering the excysted oocysts;
(d) resuspending the recovered oocysts in biphasic media;
(e) incubating the culture prepared in step (d); and
(f) recovering the oocysts.

### BRIEF DESCRIPTION OF THE DRAWINGS

FIG. 1: Sporozoites released from Cryptosporidium parvum oocysts. a. Sporozoites transformed to trophozoites, which are circular to oval in shape. b&c. Note how trophozoites aggregate together after their release from oocysts. d. Note most of oocysts detected after 24 hours were empty. 24 hrs culture of Cryptosporidium parvum oocysts in RPMI-1640 monophasic maintenance medium. Scale bar=5 μm.

FIG. 2: Meront I formed after the fusion of trophozoites released from Cryptosporidium parvum oocysts. Note the size of these meronts depend on the number of trophozoites clumped together. 72 hrs culture of Cryptosporidium parvum oocysts in RPMI-1640 biphasic maintenance medium. Scale bar=5 μm.

FIG. 3: a. Early Meront II. b. Meront II appeared as rosette with merozoites in the process of formation. C. Meront II releasing merozoites. d&e. Free merozoites released from Meront II, note some of them are spindle-shaped with pointed ends and others are circular. 8-day-old culture of Cryptosporidium parvum oocysts in RPMI-1640 monophasic maintenance medium. Scale bar=5 μm

FIG. 4: Merozoites after 8 days of culture in RPMI-1640 biphasic maintenance medium. a. Note the productivity of this system where you can see a large number of merozoites formed; also note the presence of two morphologically different types of merozoites some spindle shaped with pointed ends and others circular. Scale bar=5 μm.

FIG. 5: Sexual stages detected in Cryptosporidium parvum grown in RPMI-1640 biphasic maintenance medium for 6-7 days. a. Microgamonts with microgametes, which eventually bud off (b) from the surface. b. Early microgamont with developing microgametes where their nuclei are clearly shown. c. Late macrogamont with microgametes adhered to the surface. d. Microgametes still clumped together upon their release from microgamonts. e. Free fully developed microgametes, note the nucleus filling most of the cytoplasm. f. Fully developed macrogamont with peripheral nucleus. g. Fertilization process where you can see macro and microgametes fusing together (Mi & Ma) and a microgamete still adhering to the surface. h. Fertilization in process as macro and macrogamont (Ma & Mi) pair together. i. Free zygote (unsporulated oocyst) with big central nucleus. Scale bar=5 μm.

FIG. 6: Cryptosporidium parvum sporulated oocysts after 46 days of culturing in RPMI-1640 biphasic maintenance medium with the continuous release of sporozoites. Scale bar=5 μm

FIG. 7: Extracellular stages detected in biphasic culture after 8 days in RPMI-1640 biphasic maintenance medium. Scale bar=5 μm.

FIG. 8: Diagrammatic illustration of the life cycle of Cryptosporidium parvum in host cell free medium.

### DETAILED DESCRIPTION OF THE INVENTION

Cryptosporidium Culture Methods

The present invention provides a host-cell free method for culturing Cryptosporidium comprising the step of introducing Cryptosporidium, at a first lifecycle stage, into a host-cell free medium under conditions that enable the Cryptosporidium to progress to a second lifecycle stage.

The present invention is based on the surprising discovery that Cryptosporidium can be cultured in media in the absence of host cells. Host-cell free culture methods for Cryptosporidium are less complex than existing systems, which required the presence of different host cell lines. Furthermore, host-cell free methods avoid problems of overgrowth and aging of host cells that prevent perpetuation of the Cryptosporidium life cycle in vitro. Host cell free Cryptosporidium culture methods are also particular amenable for use in vaccine development and drug screening and are more amenable to scale up. Harvesting parasites from host cell free media is also simplified relative to the harvest of Cryptosporidium from host cell based culture media.

Whilst reference is made above to culturing Cryptosporidium and culture methods it will be appreciated that “culture” as used herein also covers methods where Cryptosporidium are maintained in a viable state in a host cell free medium, without progressing to further lifecycles. In this regard, it is possible that Cryptosporidium could be cultured in host cell containing medium and then transferred to host cell free medium and maintained in a viable state for future use.

The first and second lifecycle stages may be selected from the group consisting of: oocyst including excysted oocysts, sporozoite, trophozoite, meront I, merozoites (Type I), meront II (early), meront II (late), merozoites (type II), macrogamont, microgamete and zygote. Preferably, the first lifecycle stage is an oocyst or a sporozoite and the second lifecycle stage is an oocyst, sporozoite or a trophozoite.

The method of the present invention can be used to culture Cryptosporidium from a lifecycle stage to the oocyst stage, which effectively represents the start of the next lifecycle. Thus, the present invention also provides a host-cell free method for culturing Cryptosporidium comprising the step of introducing Cryptosporidium, at a first lifecycle stage, into a host-cell free medium under conditions that enable the Cryptosporidium to complete its lifecycle.

Culture methods that enable the Cryptosporidium to complete its lifecycle can be used to produce Cryptosporidium biomass for various uses and applications including the generation of antibodies by infecting a recipient animal and the production of immunogenic preparations such as vaccines. Thus, the present invention also provides a host-cell free method for producing Cryptosporidium biomass from an initial inoculum of Cryptosporidium comprising the steps of: (i) putting the inoculum into a host cell free medium; and (ii) culturing the Cryptosporidium to increase the Cryptosporidium biomass.

The above method can be used to maintain a viable culture of Cryptosporidium, increase Cryptosporidium biomass in general and/or be used to obtain large quantities of particular lifecycle stages. For example, if oocysts are needed then an inoculum comprising oocysts can be cultured in a host cell free medium to generate more oocysts.

The host cell free medium of the present invention can be any cell free medium that maintains Cryptosporidium or enables the progress of Cryptosporidium through its lifecycle. Such media are available commercially and can be supplemented with one or more constituents as necessary. Persons of ordinary skill in the art would be able to formulate various host cell free media based on the information herein and using their ordinary skill and knowledge.

The host cell free medium may comprise a buffered and balanced combination of inorganic salts, amino acids and vitamins. Additional constituents can be selected from the group consisting of: buffers (e.g. sodium bicarbonate, HEPES), amino acid supplements (e.g. L-glutamine), carbohydrate source (e.g. glucose), vitamins (B, B5, B complex, C), antibiotics (e.g. penicillin and streptomycin), bile (e.g. bovine bile) and serum (e.g. foetal calf serum).

The pH of the medium may be varied provide it supports the growth of the Cryptosporidium. Preferably, the pH is at or about neutral pH such as between 6.5 and 7.5. In one particular form the pH of the medium is about 7.4.

The host cell free medium may be biphasic and thus may further comprise serum that has been treated to render it viscous or semi-solid such as coagulated serum. When the host cell free medium is bi-phasic, the phases are preferably clearly separated, although one skilled in the art will appreciate that the division between the phases need not be entirely precise.

The serum used to form the second phase in the biphasic medium may be varied. Preferably, the serum is from a foetal animal, more preferably a foetal mammal and even more preferably a foetal bovine such as a cow or calf.

As indicated above, it is preferred to use oocysts as the first lifecycle stage in the method of the present invention. When oocysts are the first lifecycle stage the host-cell free method for culturing Cryptosporidium of the present invention may comprise the steps of:
(a) isolating Cryptosporidium oocysts;
(b) excysting the isolated oocysts;
(c) resuspending the excysted oocysts in a host-cell free culture medium;
(d) incubating the culture prepared in step (c) under suitable conditions; and
(e) harvesting oocysts from the medium.

Cryptosporidium for use in the method of the culture method of the present invention can be obtained from natural sources apparent to one skilled in the art. Any organism infected with Cryptosporidium is a source of Cryptosporidium. These organisms include mice, humans, bovines, or porcines that may be naturally infected or have been infected on purpose with a view to generating Cryptosporidium for further culture.

Any species of Cryptosporidium may be cultured using the present invention. Preferably, the Cryptosporidium is selected from the group comprising Cryptosporidium andersoni, Cryptosporidium parvum, Cryptosporidium muris, Cryptosporidium hominis, Cryptosporidium wrairi, Cryptosporidium felis, Cryptosporidium canis, Cryptosporidium baileyi, Cryptosporidium meleagridis, Cryptosporidium galli, Cryptosporidium serpentis, Cryptosporidium saurophilum and Cryptosporidium molnari.

Host Cell Free Media

The media used in the method of the present invention are themselves an aspect of the invention. Thus, the present invention also provides a host cell free medium capable of maintaining Cryptosporidium or enabling the progress of Cryptosporidium through its lifecycle the medium comprising a buffered and balanced combination of inorganic salts, amino acids, vitamins and additional constituents selected from the group consisting of: buffers (e.g. sodium bicarbonate, HEPES), amino acid supplements (e.g. L-glutamine), carbohydrate source (e.g. glucose), vitamins (B, B5, B complex, C), antibiotics (e.g. penicillin and streptomycin), bile (e.g. bovine bile) and serum (e.g. foetal calf serum).

The pH of the medium may be varied provide it supports the growth of the Cryptosporidium. Preferably, the pH is at or about neutral pH such as between 6.5 and 7.5. In one particular form the pH of the medium is about 7.4.

The host cell free medium may be biphasic. Thus, the present invention also provides a biphasic host cell free medium capable of maintaining Cryptosporidium or enabling the progress of Cryptosporidium through its lifecycle the medium comprising a buffered and balanced combination of inorganic salts, amino acids, vitamins and additional constituents selected from the group consisting of: buffers (e.g. sodium bicarbonate, HEPES), amino acid supplements (e.g. L-glutamine), carbohydrate source (e.g. glucose), vitamins (B, B5, B complex, C), antibiotics (e.g. penicillin and streptomycin), bile (e.g. bovine bile) and serum (e.g. foetal calf serum).

Preferably, the biphasic medium further comprises serum that has been treated to render it viscous or semi-solid such as coagulated serum. The serum used to form the second phase in the biphasic medium may be varied. Preferably, the serum is from a foetal animal, more preferably a foetal mammal and even more preferably a foetal bovine such as a cow or calf.

The biphasic medium may be prepared by preparing a first phase formed of coagulated serum and overlaying a second phase comprising a buffered and balanced combination of inorganic salts, amino acids, vitamins and additional constituents selected from the group consisting of: buffers (e.g. sodium bicarbonate, HEPES), amino acid supplements (e.g. L-glutamine), carbohydrate source (e.g. glucose), vitamins (B, B5, B complex, C), antibiotics (e.g. penicillin and streptomycin), bile (e.g. bovine bile) and serum (e.g. foetal calf serum).

As indicated above the Cryptosporidium cultured using the method of the present invention have a number of advantages relative to Cryptosporidium cultured in other systems utilising host cells. Described hereunder is a selection of the uses for Cryptosporidium cultured using the method of the present invention.

Therapeutic Applications

The culture of Cryptosporidium in the absence of host cells according to the present invention enables the production of Cryptosporidium material that is more amenable for use in therapeutic applications.

Relatively large quantities of Cryptosporidium, at any given lifecycle stage, such as trophozoites, merozoites and other extracellular gamont-like stages can be produced using the culture method described herein and then purified and isolated for further use in the development of therapeutics and immunogenic preparations such as vaccines.

Thus, the present invention also provides a method for preparing an immunogenic preparation comprising at least one Cryptosporidium antigen, the method comprising the steps of: (i) introducing Cryptosporidium, at a first lifecycle stage, into a host-cell free medium under conditions which enable the Cryptosporidium to progress to a second lifecycle stage; (ii) isolating the Cryptosporidium at the second lifecycle stage; and (iii) preparing a therapeutic preparation using the Cryptosporidium isolated from step (ii).

In the present invention, the terms “therapeutic” and “therapy” are used interchangeably and include, without limitation, the range of outcomes from prevention of disease, through maintenance of existing health levels to treatment of conditions and the curing of disease. The terms further include, without limitation, prophylaxis, alleviation of symptoms and restoration of health.

Prior to the present invention it was very difficult to obtain sufficient amounts of individual Cryptosporidium lifecycle stages to enable them to be studied for therapeutic applications. Preferably, the second lifecycle stage is an extracellular lifecycle stage and even more preferably is a trophozoite, merozoite or other extracellular gamont-like stage. These extracellular forms may be particularly useful for producing a protective therapeutic because they are likely to display antigens that are not found on intracellular forms of Cryptosporidium. Extracellular forms of Cryptosporidium are likely to be particularly useful in eliciting an IgA response in animals. An IgA response is associated with immunity and clearing of the parasite.

Thus, the present invention also provides a therapeutic composition comprising a therapeutically effective amount of Cryptosporidium cultured according to the method described herein and a physiologically acceptable carrier.

The Cryptosporidium in the therapeutic composition may comprise be a whole cell extract of one or more lifecycle stages. Alternatively, the Cryptosporidium may be a preparation of Cryptosporidium that has been treated to disrupt the cells therein. In another more processed more of the invention the therapeutic composition comprises at least one Cryptosporidium antigen that has been isolated and purified from Cryptosporidium cultured according to the present invention.

Various methods of disruption may be used, including but not limited to sonication, osmotic pressure, freezing, exposure to detergents such as sodium dodecyl sulfate (SDS), and heating. In addition to disrupting the Cryptosporidium, it may be also desirable to inactivate Cryptosporidium, or antigens produced by Cryptosporidium, before use in therapeutic compositions. Conventional techniques such as heat treatment or formalin inactivation may be used.

Therapeutic compositions may comprise one or more strains of Cryptosporidium and/or one or more antigens of Cryptosporidium. Such antigens may be used in addition to whole or sonicated protozoa or may be used in cell-free therapeutic compositions. Sonicated Cryptosporidium preparations, concentrated Cryptosporidium toxin, and other Cryptosporidium-containing preparations may be used in such therapeutic compositions.

The formulation of therapeutic compositions may include suitable pharmaceutical carriers, including adjuvants. The use of an adjuvant, for example, an alum-based adjuvant, such as aluminium hydroxide, is preferred. Commercially available adjuvants may also be used in therapeutic composition or combined with commonly available adjuvant in therapeutic compositions. For example, a preferred therapeutic composition comprises aluminium hydroxide and QUILL A (Super Fos, Copenhagen, Denmark). The precise adjuvant formulation of the therapeutic compositions will depend on the particular strain of Cryptosporidium, the species to be immunized, and the route of immunization. Therapeutic composition formulation is well-known to those skilled in the art.

Such therapeutic compositions may be used to immunize an animal susceptible to Cryptosporidium infection, including but not limited to, human, bovine, ovine, caprine, equine, leporine, porcine, canine, feline, and avian species. Both domestic and wild animals may be immunized as well as food producing animals.

The present invention further provides a method of preventing or treating a disease associated with Cryptosporidium infection comprising administering a therapeutically effective amount of Cryptosporidium cultured according to the method described herein, or a antigen isolated therefrom, and a physiologically acceptable carrier. This method is useful in, for example, dogs, cats, sheep, humans, domestic animals (especially food producing animals), avian species, and wild animals. Use in wild animals may prevent contamination of the environment, including water supplies used by humans or domestic animals.

Any convenient route of inoculation may be used to deliver the therapeutic composition and the route may vary depending on the animal to be treated, and other factors. Parenteral administration, such as subcutaneous, intramuscular, or intravenous administration, is preferred. Subcutaneous administration is most preferred for canine and feline species. Oral administration may also be used, including oral dosage forms which are enteric coated.

The schedule of administration may vary depending on the animal to be treated. Animals may receive a single dose, or may receive a booster dose or doses. Annual boosters may be used for continued protection. The age of the animal to be treated may also affect the route and schedule of administration. Administration is preferred at the age when maternal antibodies are no longer present and the animal is immunologically competent. These conditions occur at about 6 to 7 weeks of age in canine or feline species. Additionally, immunization of mothers to prevent infection of their offspring through passive transfer of antibodies in their milk is also contemplated. Treatment may be administered to symptomatic or asymptomatic animals, including animals or humans with chronic infection, and may be used to increase growth rate by alleviating such symptoms of infection as diarrhoea. Accordingly, administration of an effective amount of a therapeutic composition may increase feed conversion.

The invention further provides Cryptosporidium that have been cultured as described herein and cryopreserved. Methods of cryopreservation are described in the Examples section below. Cryopreservation solutions may comprise culture media, FBS, and a cryopreservant such as dimethyl sulfoxide (DMSO) or glycerol.

Detection of Cryptosporidium

The culture method of the present invention can be used to screen samples for Cryptosporidium. Thus, the present invention also provides a method for detecting Cryptosporidium in a sample comprising the steps of: (i) subjecting the sample to the culture method described herein; and (ii) detecting the Cryptosporidium.

The detection method of the present invention is based on increasing the amount of Cryptosporidium in the sample to a level that is more readily detectable and thus any of the culture methods described above can be applied to the detection of Cryptosporidium in a sample. Effectively, the sample becomes the inoculum of the culture method.

Thus, the present invention also provides a method for detecting Cryptosporidium in a sample comprising the steps of: (i) introducing the sample into a host-cell free medium under conditions which enable Cryptosporidium to progress to a second lifecycle stage; and (ii) detecting the Cryptosporidium.

As for the culture method described above, it is preferable that the culture method enables the Cryptosporidium to complete its lifecycle. Thus, the present invention also provides a method for detecting Cryptosporidium in a sample comprising the steps of (i) introducing the sample into a host-cell free medium under conditions that enable the Cryptosporidium to complete its lifecycle; and (ii) detecting the Cryptosporidium.

The sample may be from any source but preferably is a water sample such as a sample taken from a water source that is to be used by humans. Even more preferably, the sample is taken from a source of drinking water such as a dam, lake, river or rain catchment area.

The Cryptosporidium can be detected by any available means. For example, the sample may be viewed via a microscope or some other means that enables any Cryptosporidium in the sample to be viewed. Alternatively, the detection may be via PCR or some other lab technique designed to preferentially detect the presence of Cryptosporidium in a sample.

For some samples it may be necessary to pretreat the sample prior to culture to concentrate any Cryptosporidium in the sample. One way of doing this is to centrifuge the sample. Thus, the present invention also provides a method for detecting Cryptosporidium in a sample wherein the sample is pretreated to concentrate the Cryptosporidium therein prior to culture. In one particular embodiment of the detection method of the present invention samples are concentrated by centrifugation or any other suitable system in the art, the pellet treated for excystation and then exposed to the culture method of the present invention.

General

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in the specification, individually or collectively and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

As used herein the term “derived” and “derived from” shall be taken to indicate that a specific integer may be obtained from a particular source albeit not necessarily directly from that source.

Throughout this specification, unless the context requires otherwise, the work “comprise”, or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

The present invention will now be described with reference to the following examples. The description of the examples in no way limits the generality of the preceding description.

### EXAMPLES

### Example 1

### Host Cell Free Media

Host Cell Free Media A

A maintenance medium for Cryptosporidium culture in vitro was formed from 100 ml RPMI-1640 (Sigma, St Louis, Mo.) supplemented with 0.03 g L-glutamine, 0.3 g sodium bicarbonate, 0.02 g bovine bile, 0.1 g glucose, 25 μg folic acid, 100 μg 4-aminobenzoic acid, 50 μg calcium pantothenate, 875 μg ascorbic acid, 1% FCS, 15 mM HEPES buffer, 10,000 U penicillin G and 0.01 g streptomycin, adjusted to pH 7.4.

Host Cell Free Media B

A biphasic medium for Cryptosporidium development in vitro was generated by coagulating 5-10 ml new born calf serum in 25 cm² culture flasks for 45 minutes in a water bath at 70-80° C. The coagulated base was then overlaid with 80 ml of the maintenance media described above.

### Example 2

### Excystation of Oocysts and Culture Media Preparation

A sample of Cryptosporidium parvum (cattle genotype) (Swiss cattle C26) was obtained from the Institute of Parasitology, Zurich. The parasite was then passaged through mice and purified as previously described by Meloni, B. P. and R.C.A. Thompson (1996) J Parasitol 82:757-762. Oocysts used for the experiments were stored in PBS and antibiotics at 5° C. before use.

Cryptosporidium parvum oocysts were excysted in freshly prepared, filter-sterilised (0.22 μm filter) excystation medium composed of acidic H₂O (pH 2.5-3) containing 0.5% trypsin and incubated in a water bath at 37° C. for 20 minutes with mixing every 5 min. Thereafter, the excystation suspension was centrifuged at 2,000× g for 4 minutes at room temperature. The recovered oocysts were resuspended in maintenance medium.

### Example 3

### Oocyst Incubation and Examination of Development

Materials/Methods

Flasks (25 cm²) containing maintenance media (monophasic culture) or biphasic culture media were inoculated with 1 million excysted oocysts resuspended in 20 ml maintenance medium. The cultures were then incubated at 37° C. in a 5% CO₂ incubator.

Aliquots (10 ml) were taken from the flasks, centrifuged and the pellet examined for Cryptosporidium stages after 1, 3, 4, 8, 9, 17, 20 and 48 days of incubation. Wet mounts were prepared from the pellet and examined using Nomarski phase-contrast microscopy (Olympus BX50) and Optimas image analysis (MS-DOS operating system) for capturing images of Cryptosporidium parvum. Photographs were taken at ×400 and ×1,000 magnification.

Results

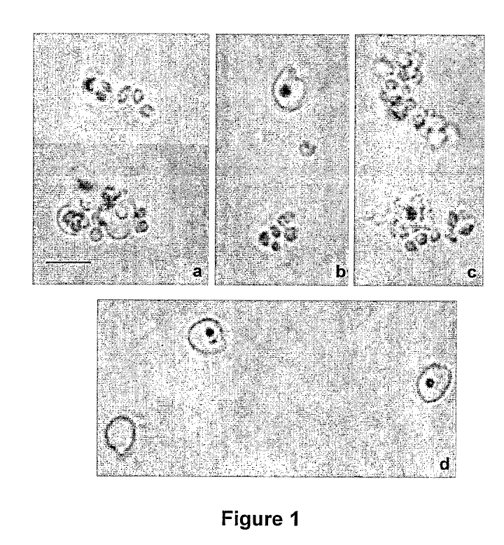

Examination of monophasic and biphasic Cryptosporidium parvum cultures after 24 hrs revealed that most oocysts had excysted (FIG. 1d) and revealed large numbers of sporozoites. Many sporozoites had transformed into circular to spindle-shaped motile trophozoites measuring 2×1.3 μm in size (FIG. 1a, b, c).

Trophozoites appeared to fuse into aggregates of two or more trophozoites and occasionally large aggregates containing 10-20 trophozoites (FIG. 1a, b, and c).

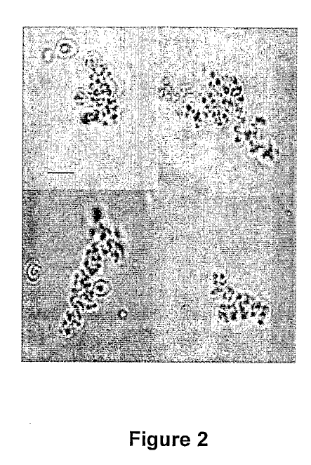

Between 48 and 72 hrs, trophozoites within aggregates developed into meronts (meront I) of variable size depending on the number of initially fused trophozoites (FIG. 2). Meront development occurred as a result of multiple mitotic divisions of the fused trophozoites.

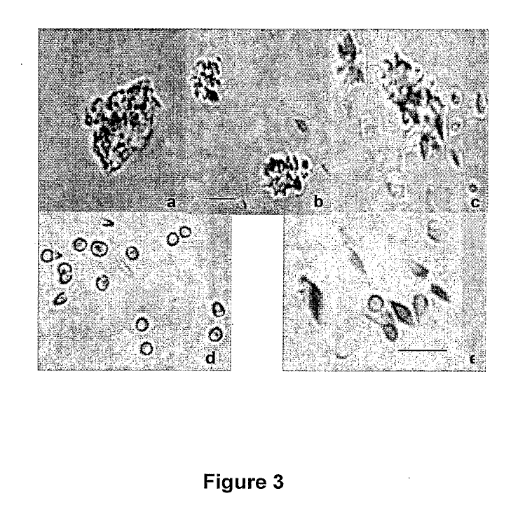

Consistent with previous studies two different types of meronts were observed (type I meronts and type II meronts) (FIG. 2 & FIG. 3).

Type I meronts appeared as grape-like aggregates as early as 48 hrs after the start of the method of Cryptosporidium culturing (FIG. 2). Merozoites released from these meronts were actively motile, circular to oval in shape and small in size (1.2×1 μm). Merozoites released from type I meronts, enlarged and clumped together to generate type II meronts.

Type II meronts, which attained a rosette-like pattern, were first detected after 3 days of culturing (FIG. 3).

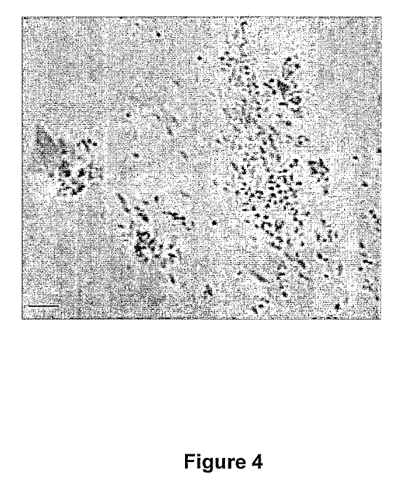

Merozoites released from type II meronts were either broadly spindle-shaped with pointed ends measuring 3.5×2 μm in size (FIG. 3b, d), or rounded to pleomorphic measuring 1.6×1.5 μm in size (FIG. 3c). After 7-8 days of culturing large numbers of actively moving merozoites continued to be released from meronts (FIG. 4).

From 9 days up to 46 days all developmental stages (sporozoites, trophozoites, merozoites, type I and II meronts and sporulated oocysts) were repeatedly observed in culture.

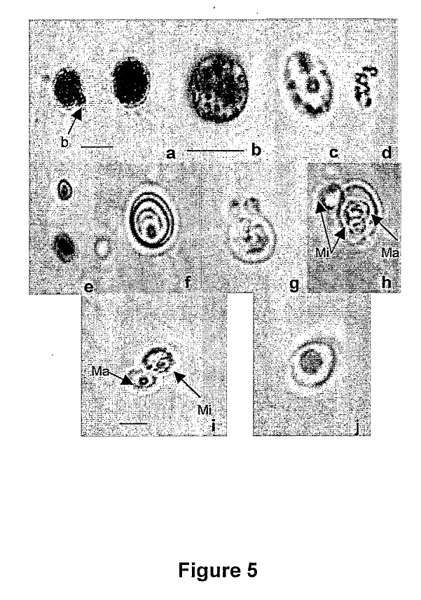

As with previous studies, it appeared that merozoites released from type II meronts developed into the sexual stages of the Cryptosporidium life cycle by transforming into macrogamonts and microgamonts (FIG. 5).

After 6 days of culturing (in biphasic medium), some merozoites released from type II meronts increased in size and developed into microgamonts (FIG. 5a-c). Microgamonts were 5.6×5 μm in size, circular in shape and appeared very dark at low magnification (FIG. 5a).

The budding of developing microgametes from the surface of the microgamont stage were evident after 6 days of culturing (FIG. 5a). At higher magnifications, microgametes can be easily differentiated from other stages by having a large number of developing microgametes on their surface. The microgamont-like stage, which appeared after 6 days of culturing (FIG. 5b,c), is similar to a Cryptosporidium baileyi microgamont. Similarities between the two stages include the circular shape and the presence of developing microgametes, which appeared as dots, which occasionally were seen to bud off from the residuum (FIG. 5b). Microgametes were observed leaving the microgamont through an opening resembling a suture was formed at the surface. Free microgametes were also observed after 7 days during the present study and again they appear similar to Cryptosporidium baileyi where the nucleus occupies most of the cytoplasm (FIG. 5d). These clumps of microgametes were detected moving freely (FIG. 5d) and fully developed microgametes, measuring 2.2×1.6 μm in size, were detected after 7 days of culturing (FIG. 5e).

Stages representing macrogamonts with characteristic peripheral nuclei were observed after 5 days and measured 5×4 μm in size (FIG. 5f and 5g). On several occasions, microgametes were observed adhering to the surface of macrogamonts and some of them were seen inside a macrogamont (FIG. 5h). Sometimes a microgamete pairing with a macrogamont was also observed (FIG. 5i).

Stages resembling zygotes and measuring 5×4 μm (FIG. 5j) were also observed after 7-8 days and had the appearance of unsporulated oocysts with a big nucleus.

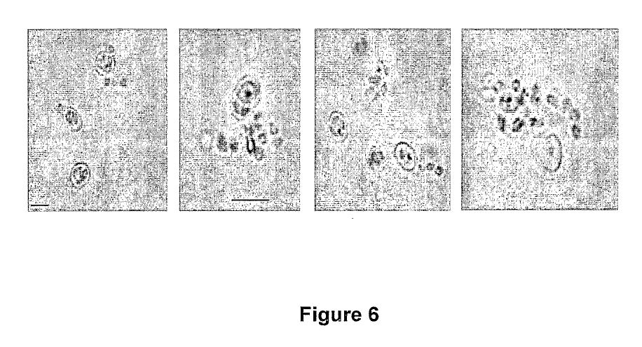

Sporulated oocysts were detected after 7-8 of culture and a significant increase in the numbers of sporulated oocysts was observed after 21 days cultivation (FIG. 6). The presence of oocysts at different stages of sporulation and the release of sporozoites from oocysts is evidence of successful fertilization and the perpetuation of the Cryptosporidium life cycle in vitro (FIG. 6).

Upon comparing the culture of Cryptosporidium in monophasic and biphasic medium two differences were noted.

First, a larger number of meronts (types I and II) were seen developing in biphasic medium, which appeared bigger in size and contained large numbers of developing merozoites than the meronts observed in monophasic medium.

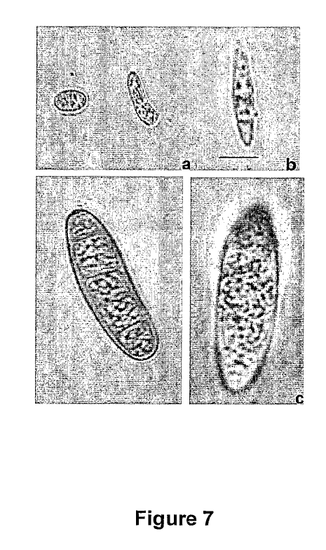

Second, the presence of gamont-like extracellular stages was not observed in monophasic medium but could be detected after 72 hrs of cultivation in biphasic medium (FIG. 7). The extracellular stages were similar to those described previously. Their size was initially small (5.3×2.3 μm) and increased with time (16.6×7.6 μm) (FIG. 7a-c).

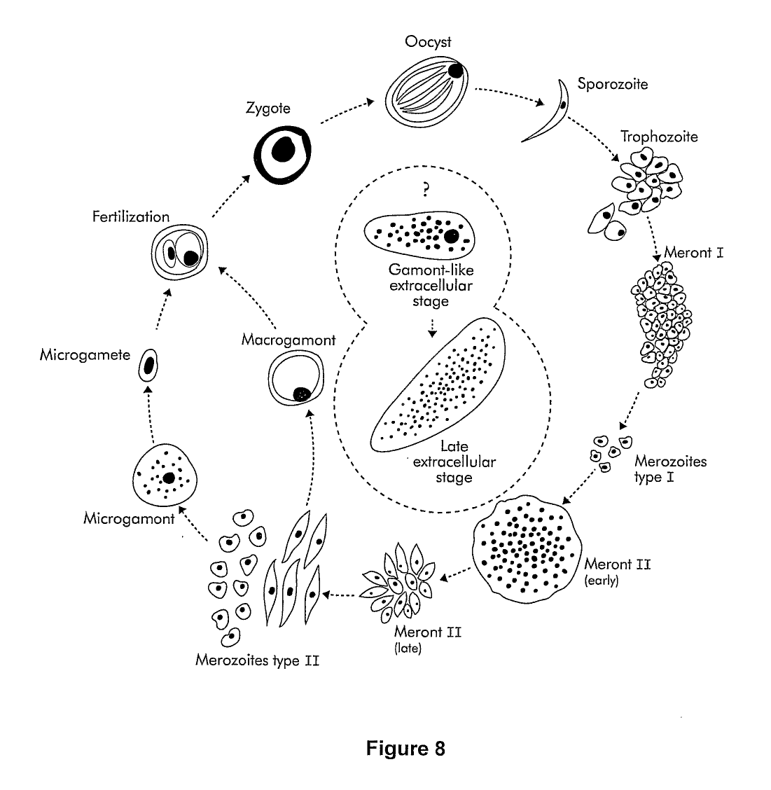

FIG. 8 shows how the life cycle of Cryptosporidium parvum proceeded in host cell free medium with developmental phases including merogony, gametogony, sporogony as well as gamont-like extracellular stages.

### Example 4

### Infectivity of Culture-derived Oocysts to Mice

Materials/Methods

Samples of maintenance medium from two 25 cm²-culture flasks containing parasites were collected from 46 day-old cultures that had been infected with 2 million oocysts of Cryptosporidium parvum (cattle genotype) purified from mice as described by Meloni, B. P. and R. C. A. Thompson (1996) J Parasitol 82:757-762. No media change was done to the sample through out the culturing period.

The culture medium was centrifuged at 2,000 g for 8 min and the pellet reconstituted in 2 ml PBS before being inoculated intragastrically into 7-8 day-old ARC/Swiss mice (100 μl/mouse). Eight days post-infection, the mice were processed for oocysts purification as described by Meloni, B. P. and R. C. A. Thompson (1996) J Parasitol 82:757-762.

Results

Oocysts collected from the 46 day-old cultures were infective to 7-8 day old ARC/Swiss mice. A yield of approximately 5×10⁶ oocysts (pooled collection from 11 mice) was obtained after purification.

### Example 5

### Cryopreservation of Cryptosporidium Oocysts Produced in Culture

Oocysts are separated from the PBS resuspension medium used following harvesting by centrifugation then resuspended in cryopreservation solution comprising 5-15% DMSO added to cell culture media comprising 10-20% FBS. Resuspended oocysts are placed on ice for several minutes, then at approximately −80° C. for 2 to 3 hours, and then stored in liquid nitrogen.

In an alternative method, the resuspended oocysts are placed directly in liquid nitrogen or in a cell freezing apparatus designed to control the freezing process.

### Example 6

### Preparing Whole Sonicated Vaccine

A whole sonicate vaccine of Cryptosporidium may be prepared using, for example, a Virsonic Cell Disrupter while maintaining the cell-culture derived parasite suspension on ice. Three 20-second bursts are generally sufficient to disrupt the parasites. The presence of intact trophozoites may be checked using a hemacytometer and an additional 20-second burst used to disrupt any intact cells.

The final protein concentration of the sonicate is determined using the BIORAD Protein Assay and adjusted to 0.75 mg/ml by the addition of sterile PBS. This solution is then mixed 1:4 with an aluminium hydroxide adjuvant for use as a vaccine preparation for immunizing animals in the following studies.

### Example 7

### Immunizing Animals with Cryptosporidium Oocysts Produced in Culture

Methods of immunizing animals against Cryptosporidium are adapted from those used to immunize against Giardia as described by Olson [U.S. Pat. Nos. 5,512,288 and 6,153,191] with minor modifications.

Two groups of five calves each are immunized (Group A) or mock-immunized (Group B) by subcutaneous injection with about 0.5 ml of an aluminium hydroxide adjuvant and about 2.5 ml of the Cryptosporidium vaccine preparation from Example 6 (Group A) or about 2.5 ml PBS (Group B). Animals may be checked for the presence of antibodies to Cryptosporidium antigens using an ELISA assay wherein purified Cryptosporidium antigen is immobilized on the ELISA plates. The presence of Cryptosporidium antibodies in the serum of immunized cattle indicates that a humoral immune response has produced antibodies to Cryptosporidium antigens in the vaccine.

### Example 8

### Challenging Inoculated Animals with Cryptosporidium

To determine whether these antibodies are protective against subsequent Cryptosporidium challenge, the immunized or mock immunized animals from Example 7 are challenged with Cryptosporidium parasites. Cryptosporidium parasites are introduced either orally or by direct intestinal inoculation.

Typically, mice are challenged with about 106 oocysts and calves are infected with about 10⁷ to about 10⁸ oocysts [see, eg, Perryman, L. E. et al (1999) Vaccine 17:2142-49; Bukhari, Z. et al (2000) Appl Envir Microbiol 66:2972-80; Sréter, T. et al (2000) Appl Envir Microbiol 66:735-738].

### Example 9

### Monitoring Animals for Clinical Evidence of Infection

Cryptosporidium-challenged animals are monitored for overt clinical signs of disease, such as soft stools, diarrhoea, weight loss, lethargy, and failure to thrive.

Faecal cyst counts are also performed daily for the duration of the infection to determine where the infected animals are shedding Cryptosporidium oocysts. Serum samples are obtained at least weekly and at post mortem for use in ELISAs to measure IgM and IgG titres.

Following euthanasia, gut samples (e.g. duodenum, jejunum, ileum) are taken for trophozoite counts, light microscopy, and electron microscopy. Mucosal scrapings, serum samples and bile are collected and stored frozen at about −80° C. for further immunological analyses and enzymatic investigations.

Reduced clinical manifestations of Cryptosporidium infection in immunized animals, compared with control animals that are not immunized, is evidence that the vaccine is effective in protecting immunized animals against Cryptosporidium infection.

### Example 10

### Enzyme Linked Immunosorbent Assay (ELISA)

Animal gut mucosal homogenates are prepared essentially as described by Olson [U.S. Pat. No. 6,153,191].

Tissue from the intestinal mucosa of infected animals is homogenized in 10% weight/volume 2 mM EDTA then stored at −80° C. Samples are then thawed and diluted about 1:1 with a solution comprising 2 mM EDTA and 1 mM PMSF. The mixture is dispersed and disrupted by five passes through an 18 G needle. Insoluble debris is pelleted by centrifugation at about 17,000× g for 20 minutes.

Supernatants containing soluble proteins are used for ELISA immediately or stored at −80° C. Polyclonal or monoclonal antibodies that detect Cryptosporidium antigen are useful in the assay. All samples are assayed in duplicate [see also, Perryman, L. E. et al (1999) Vaccine 17:2142-49]. The detection of antibodies to Cryptosporidium proteins in the serum of immunized animals is evidence of a humoral immune response to the vaccine.

Modifications of the above-described modes of carrying out the various embodiments of this invention will be apparent to those skilled in the art based on the above teachings related to the disclosed invention. The above embodiments of the invention are merely exemplary and should not be construed to be in any way limiting.

1. A host-cell free method for culturing Cryptosporidium comprising the step of introducing Cryptosporidium, at a first lifecycle stage, into a host-cell free medium under conditions which enable the Cryptosporidium to progress to a second lifecycle stage.

2. A method according to claim 1 wherein the first and second lifecycle stages are selected from the group consisting of: oocyst including excysted oocysts, sporozoite, trophozoite, meront 1, merozoites (Type 1), meront 11 (early),meront 11 (late), merozoites (type 11), macrogamont, microgamete and zygote.

3. A method according to claim 1 wherein the first lifecycle stage is an oocyst or a sporozoite and the second lifecycle stage is an oocyst, sporozoite or a trophozoite.

4. A method according to claim 1 wherein the second lifecycle stage is an oocyst.

5. A host-cell free method for culturing Cryptosporidium comprising the step of introducing Cryptosporidium, at a first lifecycle stage, into a host-cell free medium under conditions which enable the Cryptosporidium to complete its lifecycle.

6. A host-cell free method for producing Cryptosporidium biomass from an initial inoculum of Cryptosporidium comprising the steps of: (i) putting the inoculum into a host cell free medium; and (ii) culturing the Cryptosporidium to increase the Cryptosporidium biomass.

7. A method according to claim 1 wherein the host cell free medium is a buffered and balanced combination of inorganic salts, amino acids and vitamins.

8. A method according to claim 7 wherein the medium further comprises an additional constituent selected from the group consisting of: a carbohydrate source, antibiotics, bile and serum.

9. A method according to claim 1 wherein the medium has a pH at or about neutral pH.

10. A method according to claim 1 wherein the host cell free medium further comprises a second phase in the form of serum that has been treated to render it viscous or semi-solid.

11. A method according to claim 10 wherein the serum is coagulated.

12. A method according to claim 10 wherein the serum used to form the second phase is foetal calf serum.

13. A host-cell free method for culturing Cryptosporidium comprising the steps of: a. isolating Cryptosporidium oocysts; b. excysting the isolated oocysts; c. resuspending the excysted oocysts in a host-cell free culture medium; d. incubating the culture prepared in step (c) under suitable conditions; and e. harvesting oocysts from the medium.

14. A method according to claim 1 wherein the Cryptosporidium belongs to the species selected from the group consisting of: Cryptosporidium anderson, Cryptosporidium parvum, Cryptosporidium muris, Cryptosporidium hominis, Cryptosporidium wrairi, Cryptosporidium felis, Cryptosporidium canis, Cryptosporidium baileyi, Cryptosporidium meleagridis, Cryptosporidium galli, Cryptosporidium serpentis, Cryptosporidium saurophilum and Cryptosporidium molnari.

15. A host cell free medium capable of maintaining Cryptosporidium or enabling the progress of Cryptosporidium through its lifecycle, the medium comprising a buffered and balanced combination of inorganic salts, amino acids, vitamins and additional constituents.

16. A biphasic host cell free medium capable of maintaining Cryptosporidium or enabling the progress of Cryptosporidium through its lifecycle the medium comprising a buffered and balanced combination of inorganic salts, amino acids, vitamins and additional constituents.

17. A medium according to claim 15 wherein the additional constituents are selected from the group consisting of: amino acid supplements, carbohydrate source, antibiotics, bile and serum.

18. A medium according to claim 15 with a pH about neutral.

19. A medium according to claim 16 wherein the second phase comprises serum that has been treated to render it viscous or semi-solid.

20. A medium according to claim 19 wherein the serum is foetal calf serum.

21. A method for preparing an immunogenic preparation comprising at least one Cryptosporidium antigen, the method comprising the steps of: (i) introducing Cryptosporidium, at a first lifecycle stage, into a host-cell free medium under conditions which enable the Cryptosporidium to progress to a second lifecycle stage; (ii) isolating the Cryptosporidium at the second lifecycle stage; and (iii) preparing a therapeutic preparation using the Cryptosporidium isolated from step (ii).

22. A method according to claim 21 wherein the second lifecycle stage is anextracellular lifecycle stage.

23. A method according to claim 21 wherein the second lifecycle sage is a trophozoite, merozoite or otherextracellular gamont-like stage.

24. A therapeutic composition comprising a therapeutically effective amount of Cryptosporidium cultured according to claim 1 and a physiologically acceptable carrier.

25. A composition according to claim 24 comprising a whole cell extract of one or more Cryptosporidium lifecycle stages.

26. A composition according to claim 25 comprising one or more Cryptosporidium lifecycle stages that have been treated to disrupt their cellular structure.

27. A composition according to claim 24 comprising at least one isolated and purified Cryptosporidium antigen.

28. A composition according to claim 26 wherein the cellular disruption has been achieved by a technique selected from the group consisting of: sonication, osmotic pressure, freezing, exposure to detergents such as sodium dodecyl sulfate (SDS), and heating.

29. A composition according to claim 24 wherein the Cryptosporidium cells have been inactivated.

30. A method of preventing or treating a disease associated with Cryptosporidium infection in a subject comprising administering to the subject a therapeutically effective amount of a composition according to claim 24.

31. A method for detecting Cryptosporidium in a sample comprising the steps of: (i) subjecting the sample to the culture method described herein; and (ii) detecting the Cryptosporidium.

32. A method for detecting Cryptosporidium in a sample comprising the steps of: (i) introducing the sample into a host-cell free medium under conditions which enable Cryptosporidium to progress to a further lifecycle stage; and (ii) detecting the Cryptosporidium.

33. A method for detecting Cryptosporidium in a sample comprising the steps of (i) introducing the sample into a host-cell free medium under conditions which enable the Cryptosporidium to complete its lifecycle; and (ii) detecting the Cryptosporidium.

34. A method according to claim 31 wherein the sample is from a water source that is to be used by humans or animals.

35. A method according to claim 34 wherein the water source is a source of drinking water such as a dam, lake, river or rain catchment area.

36. A method according to claim 31 wherein the Cryptosporidium is detected via visual examination.

37. A method according to claim 36 wherein the visual examination is via a microscope or some other means that enables any Cryptosporidium in the sample to be viewed.

38. A method according to claim 31 wherein the Cryptosporidium is detected using PCR.

39. A method according to claim 31 further comprising the step-of pretreating the sample to concentrate any Cryptosporidium therein.

40. A method according to claim 39 wherein the pre-treatment comprises centrifugation of the sample.
